(19) United States
(12) Patent Application Publication
(10) Pub. No.: US 2023/0404589 A1
(43) Pub. Date: Dec. 21, 2023

(54) EMBOLIC COIL DELIVERY SYSTEMS AND COMPONENTS

(71) Applicant: Cook Medical Technologies LLC, Bloomington, IN (US)

(72) Inventors: Jan Kristoffersen, Næstved (DK); Jens Kold, Jyllinge (DK); Louise Thrysøe Ekstrom, 5370 Mesinge (DK); Kim Møgelvang Jensen, Frederiksberg (DK); Betina Hansen, Borup (DK); Benjamin Christian Waedeled, Nyborg (DK); Anders Daniel Hviid Jakobsen, Dragør (DK)

(73) Assignee: Cook Medical Technologies LLC, Bloomington, IN (US)

(21) Appl. No.: 18/338,819

(22) Filed: Jun. 21, 2023

Related U.S. Application Data
(60) Provisional application No. 63/354,078, filed on Jun. 21, 2022.
(60) Provisional application No. 63/354,093, filed on Jun. 21, 2022.

(51) Int. Cl. A61B 17/12

(52) U.S. Cl. CPC A61B 17/12154; A61B 17/12113; A61B 2017/12054

(57) ABSTRACT

Described are systems for delivering embolic coil devices, and components useful in such systems. The systems include a flexible elongate delivery shaft having a distal region and an embolic coil device detachably connected to the distal region of the elongate delivery shaft. In some forms, as detachably connected, at least some of the coil windings of the embolic coil device, such as those in a proximal segment of the coil windings, are in a resiliently longitudinally compressed condition. When the embolic coil device is detached from the delivery shaft, the coil windings resiliently move to a longitudinally extended condition, where the coil windings in the longitudinally extended condition are more open than they are in the resiliently longitudinally compressed condition. In some forms, the systems have a coil detachment interface at a distal region of a flexible elongate delivery shaft. The coil detachment interface includes a distal tubular segment and a bridge segment proximal of the distal tubular segment. The bridge segment includes a bridge wall or other bridge component defining an upper surface, a lower surface, a proximally-facing edge spanning between the upper surface and the lower surface, and a distally-facing edge spanning between the upper surface and the lower surface. The bridge segment further defines a lumen between the lower surface of the bridge wall or other bridge component and a bottom wall of the bridge segment. Also described are embolic coil devices and bridge members configured for use in such systems.

### REFERENCE TO RELATED APPLICATIONS

The present application claims the benefit of priority of U.S. Provisional Patent Application Ser. No. 63/354,078 filed Jun. 21, 2022 and of U.S. Provisional Patent Application Ser. No. 63/354,093 filed Jun. 21, 2022, each of which is hereby incorporated by reference in its entirety.

### BACKGROUND

The present disclosure relates generally to systems for implanting embolic coil devices for establishing an embolus or vascular occlusion in a patient vessel, and to devices and components useful in such systems.

Embolic coil devices are used to treat a variety of medical conditions, including for example to treat intravascular aneurysms. An embolic coil typically takes the form of a soft, helically wound coil formed by winding wire (e.g. a platinum or platinum alloy wire) about a primary mandrel. In some known forms, the thus-formed coil is then wrapped around a larger, secondary mandrel, and heat treated to impart memory for a secondary shape. The secondary shape can also be imparted by cold forming. Upon delivery from a tubular device such as a catheter to a treatment site, the coil will transition to or toward its more convoluted secondary shape.

Various arrangements are known for detaching the coil from the delivery shaft, including notably electrolytic and mechanical detachment arrangements. Many known mechanical detachment arrangements have a delivery shaft defining a lumen and a pull wire (also termed a “release” wire) that interfaces with a feature of the coil device and that can be moved proximally in the shaft lumen to cause detachment of the embolic coil device.

There remain needs for embolic coil delivery systems that incorporate detachment features or mechanical detachment arrangements that are conducive to manufacture and that are easy and reliable in use. Aspect of the present disclosure are address to these needs.

### SUMMARY

In some aspects, provided are systems for delivering an embolic coil device. The systems include a flexible elongate delivery shaft having a distal region. An embolic coil device is detachably connected to the distal region of the elongate delivery shaft with coil windings of the embolic coil device in a resiliently longitudinally compressed condition. Upon detachment of the embolic coil device from the elongate delivery shaft the coil windings resiliently move to a longitudinally extended condition, where the coil windings in the longitudinally extended condition are more open than they are in the resiliently longitudinally compressed condition. The coil windings involved can represent some or all of the coil windings of the embolic coil device. In some forms, the coil windings involved are only some of the overall coil windings, and provide a first segment of the coil windings operable as above stated, the first segment preferably being located in a proximal region of the coil device. The first segment can be flanked by a proximal segment having coil windings that are relatively closed (more narrowly spaced) as compared to those of the first segment. In addition or alternatively, the first segment can be flanked by a distal segment having coil windings that re relatively closed as compared to those of the first segment.

In some additional aspects, provided are systems for delivering an embolic coil device. The systems include a flexible elongate delivery shaft defining a lumen and having a distal region and a coil detachment interface at the distal region of the elongate delivery shaft. The coil detachment interface includes a distal tubular segment and a bridge segment proximal of the distal tubular segment. The bridge segment includes a bridge component such as a bridge wall having an upper surface and a lower surface, and the bridge segment defines a lumen between a lower surface of the bridge wall or other bridge component and a bottom wall of the bridge segment. An embolic coil device is detachably connected to the distal region of the elongate delivery shaft, optionally with a proximal end of coil windings of the embolic coil device positioned at a location distal of the distal tubular segment of the elongate delivery shaft. A pull wire extends through the lumen of the elongate delivery shaft and interfaces with a retention member of the embolic coil device. Proximal retraction of the pull wire causes detachment of the embolic coil device from the elongate delivery shaft. In some forms, the distal tubular segment and the bridge segment are provided by a bridge member, which can also have a proximal tubular segment proximal of the bridge segment and/or can define at least one sidewall opening positioned to provide an opening portion proximal and proximate to a proximal surface of the bridge wall or other bridge component that provides the bridge segment. The bridge member can be a monolithic structure formed from a single length of tube, and can be attached to the distal end of a length of metal hypotube that provides all or part of the remainder of the elongate delivery shaft. In some forms, the bridge wall defines a proximally-facing edge spanning between the upper surface and the lower surface of the bridge wall, and a distally-facing edge spanning between the upper surface and the lower surface of the bridge wall.

In other aspects, provided are bridge members that are useful in a detachment interface of an embolic coil device delivery system. The bridge members include a distal tubular segment, a proximal tubular segment, and an intermediate segment between the distal tubular segment and the proximal tubular segment. The intermediate segment includes a bridge component such as a bridge wall defining an upper surface and a lower surface. The intermediate segment also defines a lumen between the lower surface of the bridge wall or other bridge component and a bottom wall of the intermediate segment. The bridge member can be a monolithic structure and can be formed from a single length of tube, and can have various configurations for the bridge wall including generally planar or multi-curved bridge walls. In some forms, the bridge wall has a proximally-facing edge spanning between the upper surface and the lower surface of the bridge wall, and a distally-facing edge spanning between the upper surface and the lower surface of the bridge wall.

In other aspects, provided are embolic coil devices configured for use in the systems described above and elsewhere herein.

In yet other aspects, provided are methods for making and methods for using systems and embolic coil devices as described above and elsewhere herein.

Still further embodiments, as well as features and advantages of embodiments described herein, will be apparent to persons skilled in the relevant field from the descriptions herein.

### BRIEF DESCRIPTION OF THE DRAWINGS

FIG. 1 provides an illustration of one embodiment of an embolic coil delivery system herein received within a catheter with portions of the catheter cut away to display system components therein.

FIG. 2 provides an enlarged view of section 2S of FIG. 1.

FIG. 3 provides a cross-sectional view of components in the region of a delivery shaft/coil device interface of the system of FIG. 1.

FIG. 4 provides a side view of a bridge member forming the distal-most segment of a delivery shaft of the system of FIG. 1.

FIG. 5 provides a right end view of the bridge member of FIG. 4.

FIG. 6 provides cross-sectional view taken along line XS6-XS6 of FIG. 4 and viewed in the direction of the arrows.

FIG. 7 provides a perspective view of the bridge member of FIG. 4.

FIG. 8 provides an illustration of the embolic coil device and the distal end of the delivery shaft of the system of FIG. 1 prior to associating the delivery shaft and embolic coil device with a detachable connection.

FIG. 9 provides an enlarged view of section 9S of FIG. 8.

FIG. 10 provides an enlarged view of section 10S of FIG. 8.

FIG. 11 provides an illustration of the embolic coil device and the distal end of the delivery shaft of the system of FIG. 1 after associating the delivery shaft and embolic coil device with a detachable connection.

FIG. 12 provides an enlarged view of section 12S of FIG. 11.

FIG. 13 provides a side view of one alternative bridge member for forming the distal-most segment of a delivery shaft of an embolic coil delivery system herein.

FIG. 14 provides a right end view of the bridge member of FIG. 13.

FIG. 15 provides a top view of the bridge member of FIG. 13.

FIG. 16 provides a cross-sectional view taken along line XS16-XS16 of FIG. 15 and viewed in the direction of the arrows.

FIG. 17 provides a perspective view of the bridge member of FIG. 13.

FIG. 18 provides a side view of another alternative bridge member for forming the distal-most segment of a delivery shaft of an embolic coil delivery system herein.

FIG. 19 provides a perspective view of the bridge member of FIG. 18.

FIG. 20 provides an illustration of one embodiment of an embolic coil delivery system incorporating the bridge member of FIG. 18.

FIG. 21 provides an enlarged view of section S21 of FIG. 20.

### DETAILED DESCRIPTION

Reference will now be made to certain embodiments, some of which are illustrated in the drawings, and specific language will be used to describe the same. It will nevertheless be understood that no limitation of the scope of the disclosure is thereby intended. Any alterations and further modifications in the described embodiments and any further applications of the principles as described herein are contemplated as would normally occur to one skilled in the art to which this disclosure relates.

As disclosed above, aspects of the present disclosure relate to systems for delivering an embolic device to a patient, and related devices, components and methods. The systems can include an elongate delivery shaft and an embolic device, for example an embolic coil device, detachably connected to a distal region of the delivery shaft, wherein the medical device can be detached from the delivery shaft by proximal movement of a pull wire.

As used herein, the term “proximal” means close to the operator and the term “distal” means away from the operator.

Spatially relative terms such as “lower”, “upper”, “under”, “over”, “above” and the like may be used to describe and element's and/or feature's relationship to another element(s) or feature(s), for example as depicted in the Figures. It will be understood that the spatially relative terms are intended to encompass different operations of the system, device or component in use in addition to the orientation described or depicted in the Figures. For example, if a device or component as depicted in the Figures is inverted, an element that is shown and described as “upper” would then be oriented as “lower”.

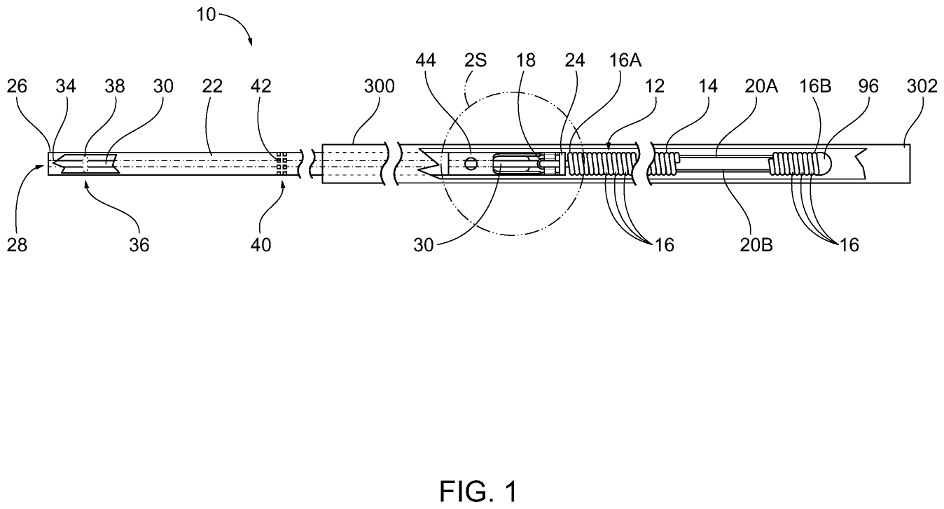

Turning now to the Figures, FIG. 1 illustrates a system 10 for delivering an embolic device 12 to a vascular space such as an aneurysm, received within the lumen of a catheter 300. The embolic device 12 may be formed as an embolic coil device 14 having a plurality of coil windings 16 extending from a proximal end 16A to a distal end 16B. Typically, the coil windings 16 are made from wire, with the wire usually made of a metal such as a platinum, a platinum alloy, or a superelastic metal alloy (e.g. a superelastic nickel/titanium alloy such as nitinol). The diameter of the wire forming the coil windings 16 may be in the range of about 0.005 mm to about 0.15 mm. The coil formed by windings 16 may have an outer primary diameter of between about 0.075 and about 1 mm and in some forms will have an outer primary diameter of between about 0.2 mm and about 0.5 mm, especially those coil devices 14 intended for use in neurovascular applications.

The axial length of the embolic coil device 14 will usually fall in the range of around 0.5 to around 100 cm, more usually around 2 to 40 cm. It will be understood, however, that other axial lengths may be employed depending on the application. As well, it will be understood that the embolic coil device 14 may have a thrombogenic material such as fibers (not shown) connected to the coil windings 16 to enhance its thrombogenicity, and that other embolic coil devices describe herein may also similarly include such a thrombogenic material.

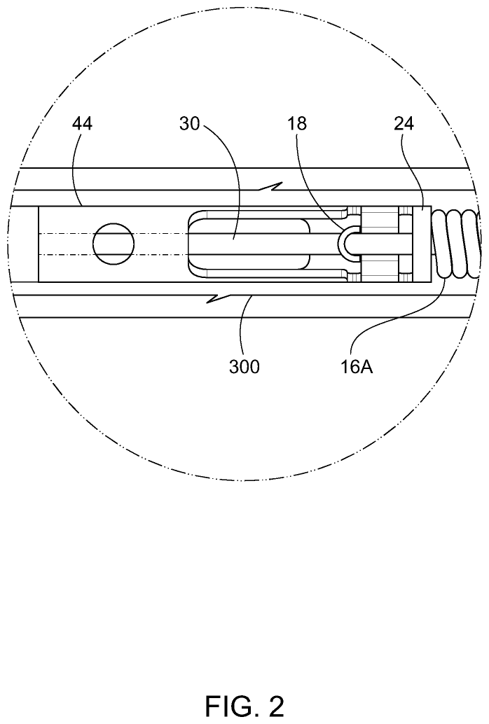

With continued reference to FIG. 1 together with FIGS. 2, 8, 9 and 10, the coil device 14 includes a proximal retention member in the form of a loop 18. The retention member in the illustrated embodiment is formed as loop 18 by a length of filament 20 that is attached to the coil device 14. The filament 20 can be formed from a suitable polymeric or metal material, for example, and may in some variants have a diameter of about 0.01 to about 0.1 mm. A suture material, especially a durable (non-bioabsorbable) suture material, may be suitably used as filament 20. The loop 18 provides two filament segments 20A and 20B that extend into and are attached to the coil windings 16, as discussed further below. While the specifically illustrated retention member is a loop 18, other retention members are known and can be used, including as examples balls, hooks, clasps, or other similar elements that can facilitate a detachable connection of the embolic coil device to the delivery shaft.

The delivery system 10 also includes a flexible elongate delivery shaft 22 having a distal end 24, a proximal end 26, and a lumen 28 therebetween. The delivery shaft 22 may be formed from any suitable material, including as examples polymeric materials, metal materials, or combinations thereof. Metal hypotube materials such as stainless steel hypotube or nitinol hypotube material may be used. As illustrative polymer materials, flexible and lubricious materials such as polyimide, polytetrafluoroethylene (PTFE), polyetheretherketone (PEEK), fluorinated ethylene propylene (FEP), or the like, may be used. As well, combinations of different metal hypotube materials, such as a combination including a first stainless steel hypotube segment attached (e.g. welded) to a second, different stainless steel hypotube segment can typically be used. For example, the second stainless steel hypotube segment may be positioned distal to, and may be more flexible and/or shorter than, the first hypotube segment. This can provide an overall delivery shaft 22 that is more flexible in its distal region than in its proximal region. Combinations of other materials that provide a more flexible distal region and a less flexible proximal region can also be used in some forms.

Metal hypotube materials, and thus the segments of the delivery shaft 22 made from them, can in some aspect have internal diameters in the range of about 0.5 to about 0.9 mm and/or external diameters in the range of about 0.6 to about 1 mm. In some preferred small diameter forms, such hypotube materials will have internal diameters in the range of about 0.1 to about 0.4 mm and/or external diameters in the range of about 0.2 to about 0.5 mm. The wall thickness of the hypotube materials, between the internal and the external diameter, can range from about 0.05 to about 0.25 mm.

The delivery shaft 22 will generally have a length between the distal end 24 and the proximal end 26 that permits the shaft to be advanced intravascularly to the target site for delivery of the coil device 14 while leaving the proximal end 26 positioned outside of the patient's body. For example, the delivery shaft 22 can have a length in the range of about 1 to about 2 meters.

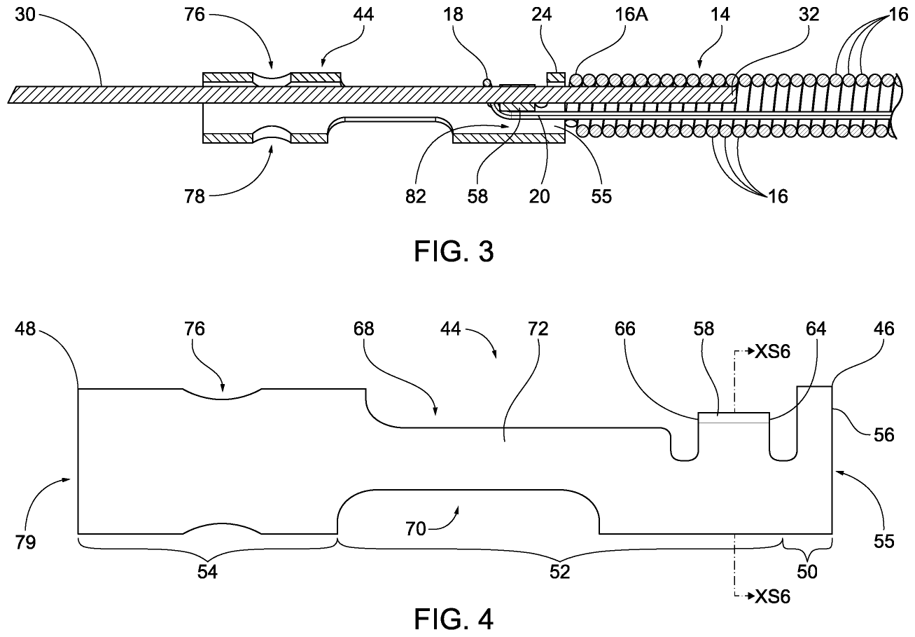

With reference particularly to FIGS. 1, 2 and 3, the system 10 also includes an elongate pull wire 30 disposed within the lumen 28 of the delivery shaft 22. The elongate pull wire 30 has a distal end 32 (see FIG. 3) and a proximal end 34 (see FIG. 1). The elongate pull wire 30 is typically formed from a flexible material that provides sufficient column strength to avoid breakage or kinking during use of the system 10. For example, the elongate pull wire 30 may be formed from one or more polymeric or metal materials, or combinations thereof. The pull wire 30 may be formed from a metal such as nitinol, titanium, titanium alloy, platinum, stainless steel or the like. The pull wire 30 has a diameter that is less than the diameter of the lumen 28 of the elongate delivery shaft 22. In one exemplary embodiment, the pull wire 30 is formed from nitinol, optionally coated with a lubricious polymer such as polytetrafluoroethylene (PTFE) and has an outer diameter of about 0.1 mm.

The system 10 may also include an arrangement for protecting against unintended proximal retraction of the pull wire 30 that may cause unintended release of the coil device 14. As illustrated in FIG. 1, at a first location 36, the delivery shaft 22 is attached to the pull wire 30. This attachment can, for example, be provided by an amount of adhesive 38, although other attachments such as welds or frictional engagements provided by one or more crimps of the shaft 22 against the outer surface of the pull wire 30 may be used. This attachment fixes the relative positions of the shaft 22 and pull wire 30. At a second location 40 distal of the first location 36, the delivery shaft 22 has a section at which the shaft 22 is configured to selectively break and separate in response to bending forces applied across the first location 36 (as compared to the sections of shaft 22 that flank location 36 on either side). After this, the separated proximal piece of shaft 22, which is attached the pull wire 30 at location 36, can be moved (e.g. pulled) proximally so as to retract pull wire 30 proximally within lumen 28 in a sliding movement and thereby cause detachment of the embolic coil device 14, as will be discussed in more detail below. In this manner, the system 10 can be used to advance the embolic coil device 14 to a desired target location in the vasculature of the patient, after which the shaft 22 can be broken and separated at location 40 and the separated proximal piece of shaft 22 can be pulled proximally to cause detachment of the coil device 14.

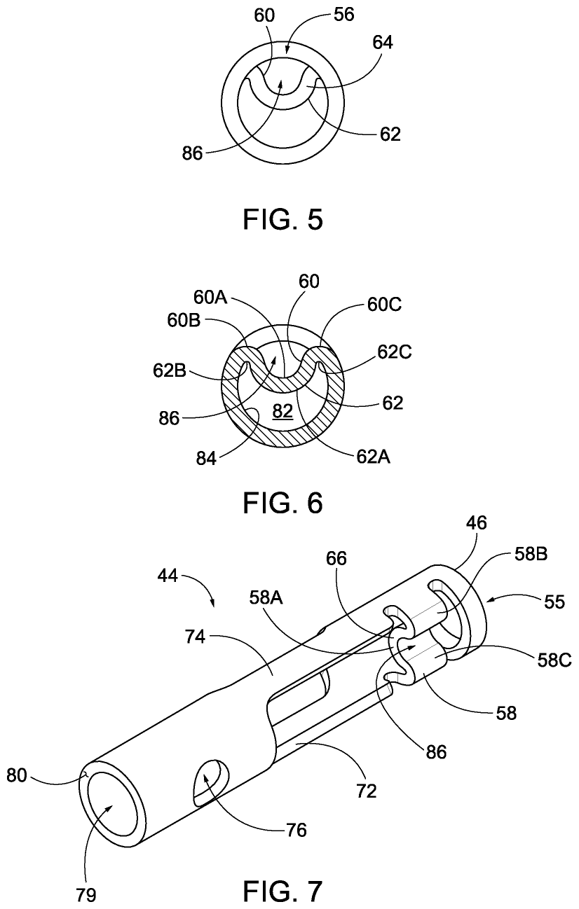

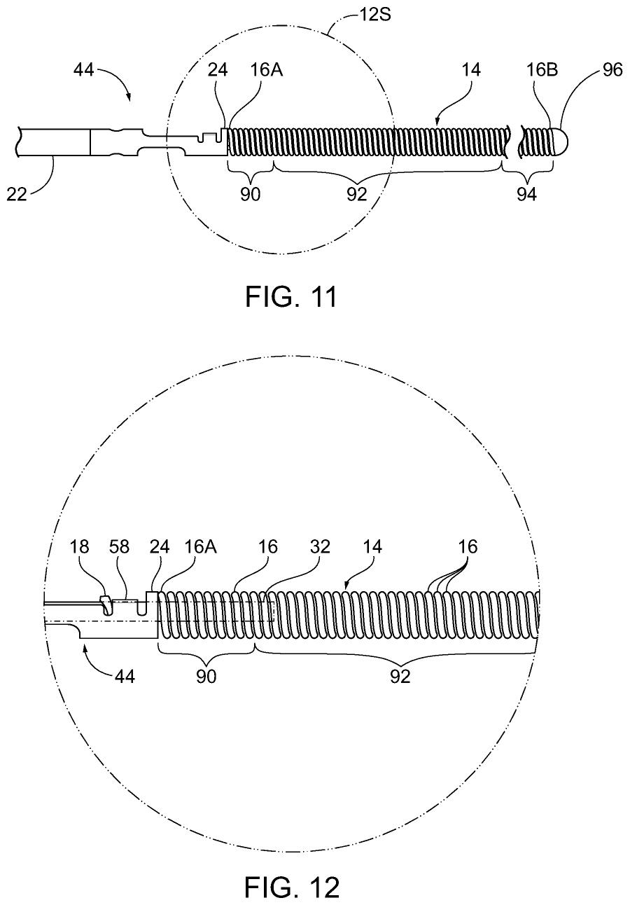

With reference now to FIGS. 1 to 12 together, embodiments will be described in which the distal region of the shaft 22 that provides an interface for detachable connection and release of the embolic coil device 14 is beneficially configured. In the illustrated embodiment, a bridge member 44 provides the distalmost segment of the shaft 22. Bridge member 44 extends from a distal end 46 that provides the distal end 24 of shaft 22, to a proximal end 48. Bridge member 44 generally has three segments, a distal segment 50, an intermediate or bridge segment 52, and a proximal segment 54. In the illustrated embodiment, the distal segment 50 is provided as a tubular segment that defines a lumen 55 and a distally-facing full circumferential end surface 56. Full circumferential end surface 56 advantageously provides a complete 360 degree surface, preferably an annular surface with an outer circular circumference and an inner circular circumference, for interfacing with (e.g. abutting) the proximal end 16A of the coil windings 16 of the coil device 14. It will be understood that other embodiments can have other end surface configurations for the bridge member 44, including those in which the end surface does not provide a full 360 degree circumferential end surface that faces distally; however, a substantial distally facing end surface, for example one that provides a coplanar end surface for at least 270 degrees or at least 320 degrees around a circumference (such surface extent being continuous in some forms and discontinuous in other forms), will be advantageous and is desirably provided. The end surface 56 may have an outer circumference at least as great as, and potentially greater than, the primary outer diameter of the coil formed by coil windings 16 at the proximal end 16A. Intermediate segment 52 of bridge member 44 provides a transverse bridge component, preferably in the form of a bridge wall 58, that extends transverse to, and preferably perpendicular to, the longitudinal axis of the delivery shaft 22. Bridge wall 58 can, as depicted, be rectangularly-shaped in longitudinal cross-section (see e.g. FIG. 3). In other embodiments, the bridge wall 58 or other transverse bridge component can have another shape in longitudinal cross-section, for example another polygonal or a rounded shape in longitudinal cross-section. Bridge wall 58 defines an upper surface 60, a lower surface 62, a distally-facing edge 64, and a proximally-facing edge 66. Edges 64 and 66 span between the upper and lower surfaces 60 and 62 of the bridge wall 58. Intermediate segment 52 also defines at least one sidewall opening, for example bridge-side opening 68 and/or an opposite side opening 70 which may, for example, provide access to facilitate assembly steps during the manufacture of system 10 and in particular the coordination and assembly of the loop 18 of the coil device 14 secured around the pull wire 30 at a location proximal of the bridge wall 58 or other transverse bridge component. To facilitate these purposes, the at least one side opening, e.g. opening 68 and/or opening 70, can have a substantial maximum width in a direction perpendicular to the longitudinal axis of bridge member 44, for example with such width being at least 40%, or at least 50%, or at least 60%, of a value equal to the corresponding width of the bridge member that longitudinally coextends with the opening(s). In addition or alternatively, the at least one side opening (e.g. opening 68 and/or 70) can have a substantial maximum length in the direction of the longitudinal axis of the bridge member 44, for example with such length being at least equal to, and in some forms greater than (for example at least 150% of or at least 200% of) a value equal to the maximum width of the opening in a direction perpendicular to the longitudinal axis of the bridge member 44. As well, the at least one side opening (e.g. opening 68 and/or opening 70) can be positioned to have a portion of the opening(s) occurring proximal and proximate to the proximal surface of the bridge wall 58 or other transverse bridge component, for example wherein such portion of the opening is proximal of the proximal surface of the bridge wall 58 or other transverse bridge component by a distance no greater than 200%, or in some forms no greater than 100%, of a value equal to the width of the bridge member 44 that longitudinally coextends with the opening(s). Upper surface 60 of bridge wall 58 can, as in the depicted embodiment, provide an outermost (upper) surface of the bridge member 44 in the longitudinal segment of the bridge member 44 (or generally the shaft 22) in which the bridge wall 58 occurs (there is no tubular wall of bridge member 44 enclosing bridge wall 58). Opposite wall portions 72 and 74 of intermediate segment 52 span between openings 68 and 70.

The proximal segment 54 of bridge member 44 is generally tubular in shape. In some embodiments, the proximal segment 54 may define first and second side wall openings 76 and 78 opposite one another. Openings 76 and 78 in the illustrated embodiment can provide a means to mount bridge member 44 during stages of its manufacture or its handling in assembly steps, although openings 76 and 78 or other similar openings may also provide visual sight line(s) or other access to the interior of bridge member 44, for example. It will be understood that the opening 68 and/or the opening 78 can be absent in other embodiments. Proximal segment 54 provides the proximal end 48 of bridge member 44 and defines a lumen 79 and a proximally-facing full circumferential end surface 80. Full circumferential end surface 80 advantageously provides a complete 360 degree surface for an end-to-end attachment of the bridge member 44 to the adjacent segment of delivery shaft 22, for example provided by an adjacent length of metal (e.g. stainless steel) hypotube or other tube material. This attachment in some forms can be a welded attachment.

The bridge member 44 defines a bridge wall lumen 82 between the lower surface 62 of bridge wall 58 and an inner tube surface 84 of the bridge member 44. The bridge wall lumen 82 generally has a smaller cross-sectional lumen area than the lumen 55 of the distal segment 50 of the bridge member 44 and the lumen 79 of the proximal segment 54 of the bridge member 44. The bridge wall lumen 82 can manage the position of the filament material 20 passing through the lumen, as in the illustrated embodiment. In other embodiments, the bridge wall lumen 82 could be used to manage the position of the pull wire 30 when it is arranged to pass through the lumen 82, for example where filament 20 may pass over the upper surface 60 of bridge wall and position loop 18 around pull wire 30 in an orientation generally opposite that depicted. Bridge wall 58 and lumen 82 can be appropriately shaped and/or sized for these and other arrangements. Generally speaking, the bridge wall 58 separates the bridge wall lumen 82 from an area occurring above the upper surface 60 of the bridge wall 58, so that different component portions of the system (e.g. portions of a pull wire or a retention member) can be maintained separated from one another by the bridge wall 58, with one of the components passing through lumen 82 and the other passing above and potentially in contact with the upper surface 60 of bridge wall 58.

In the illustrated embodiment, the bridge wall 58 of bridge member 44 has a non-planar shape. In particular, the bridge wall 58 is a multi-curved wall section with a central curved segment 58A (see e.g. FIG. 7) that curves radially inwardly that is flanked by first and second curved segments 58B and 58C that curve radially outwardly. Thus, the central curved segment 58A defines a concavely-curved surface portion 60A (see e.g. FIG. 6) of upper surface 60 of bridge wall 58 and the first and second curved segments 58B and 58C define convexly-curved surface portions 60B and 60C of upper surface 60. On the other hand, the central curved segment 58A defines a convexly-curved surface portion 62A of lower surface 62 of bridge wall 58 and the first and second curved segments 58B and 58C define concavely-curved surface portions 62B and 62C of outer surface 60. In the illustrated embodiment, the central curved segment 58A defines a longitudinal channel 86 for slidably receiving and the pull wire 30.

The bridge member 44 and other bridge members disclosed herein can be a monolithic structure. Such bridge members can be formed from a single length of tube, for example a single length of metal (e.g. stainless steel) hypotube, which can have the same dimensional and/or other features described herein for hypotube materials of the delivery shaft 22. The hypotube or other tube material used to form the bridge member 44 and other bridge members herein can be cut and formed to provide the disclosed bridge member features. Forming operations such as bending and stamping may be used, as examples. Bridge members formed from a single length of tube are advantageous in manufacture in that there is no need to assemble and attach a plurality of parts together to make the bridge members, which can be dimensionally small and thus present handling and attachment challenges. Bridge member 44 and other bridge members described herein may also be manufactured using three-dimensional metal printing to reduce or eliminate the need for metal forming processes such as bending or stamping, while still providing a monolithic structure bridge member. It will be understood that bridge member 44 or other bridge members described herein may also be manufactured by connecting (e.g. welding) multiple metal or other pieces together. Bridge member 44 and other bridge members described herein can in certain aspects have a longitudinal length of no greater than about 10 mm, or no greater than about 5 mm, and typically in the range of about 1 mm to about 5 mm or about 1 to about 3 mm.

In a detachment operation, the pull wire 30 is pulled proximally within the lumen 28 of delivery shaft 22 (e.g. after breaking the shaft 22 at location 40 when the above-discussed release arrangement is present) until the distal end 32 of pull wire 30 passes through and proximally beyond the loop 18. This releases the loop 18 to movement distally through bridge wall lumen 82 and out of the distal end 46 of bridge member 44. The coil device 14 is thereby detached from the delivery shaft 22. In one mode of use, prior to detachment of the coil device 14, the shaft 22 can be used to push the coil device 14 out of the distal end 302 of the catheter 300 at the target vascular site for delivery, with the coil device contacting vascular walls at the site and lodging in place. This operation may position the bridge member 44 distally beyond the distal end 302 of the catheter 300. The proximal movement of the pull wire 30 and consequent detachment of the coil device 14 can then occur with the bridge member deployed beyond the distal end 302 of the catheter 300.

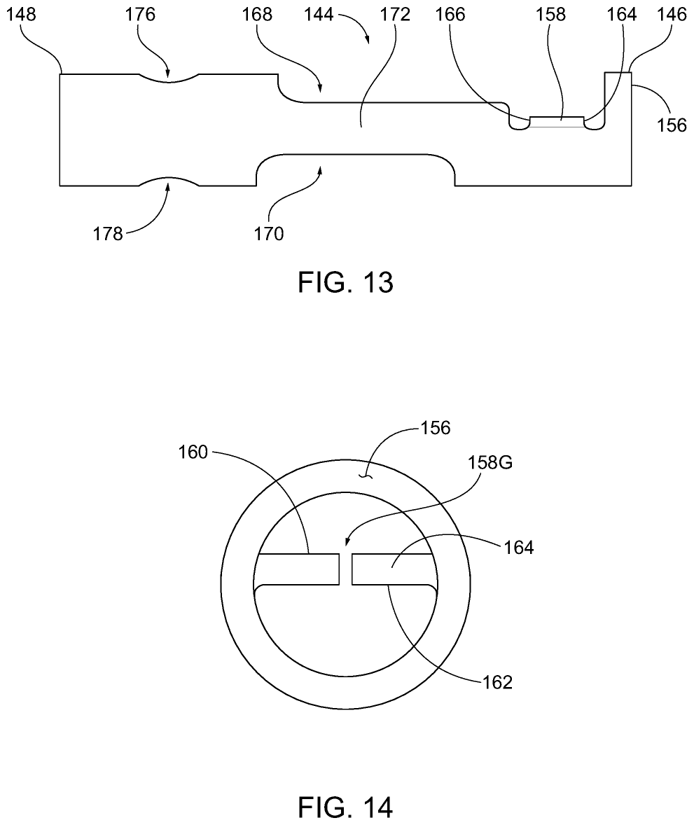

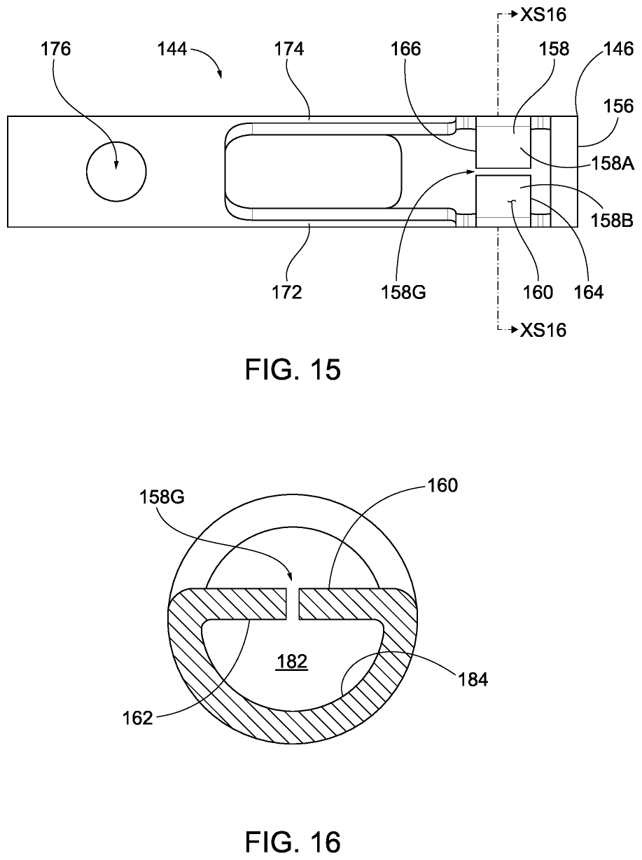

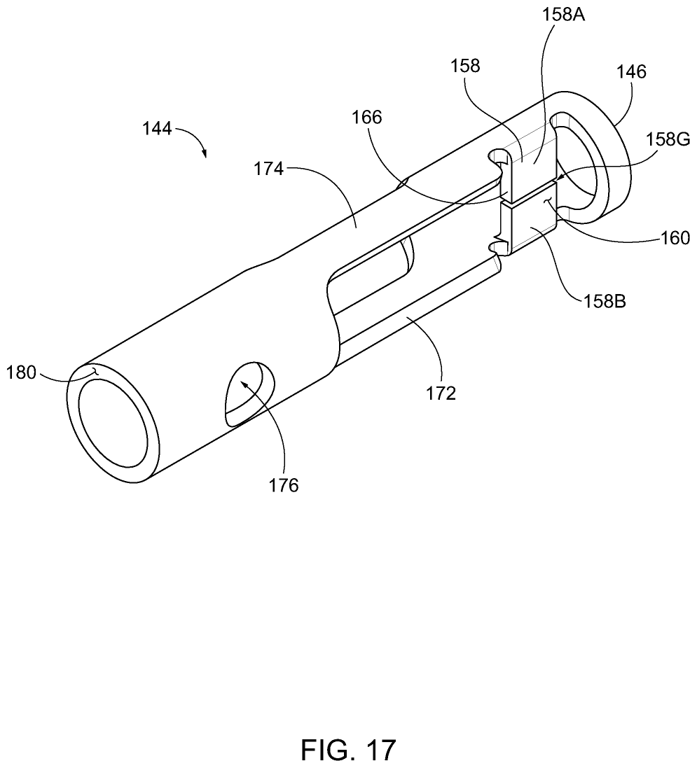

Other bridge member structures and delivery systems incorporating them are contemplated herein. Referring now to FIGS. 13 to 17, shown is one alternative embodiment of a bridge member 144. Bridge member 144 can have features that correspond to the features of bridge member 44, and such features of bridge member 144 are correspondingly numbered to those of bridge member 44 except in the 100 series (e.g. 144 vs. 44, 146 vs. 46, etc.) and thus the corresponding descriptions will not be repeated here. Bridge member 144 differs from bridge member 44 in respect of the bridge wall 158 and in respect of the bridge wall lumen 182 in part defined by the bridge wall 158. In particular, bridge wall 158 is provided as a generally straight wall extending perpendicular to the longitudinal axis of bridge member 144. Bridge wall 158 is provided by a first bridge wall piece 158A and a second bridge wall piece 158B separated by a gap 158G. Bridge wall lumen 182 of bridge member 144 has a generally straight upper wall provided by the lower surface 162 of bridge wall 158, which adjoins a continuously curved wall providing curved tube surface 184. Lower surface 162 and tube surface 184 define the shape of bridge wall lumen 182, which can generally be “D” shaped.

As noted above, bridge member 144 can be substituted for bridge member 44 in the embodiments disclosed in FIGS. 1 to 12. In doing so, the pull wire 30 will be positioned above and typically against the upper surface 160 of bridge wall 158, and the filament segments 20A and 20B of filament material 20 attached to the embolic coil device 14 will extend through the distal end of bridge member 144 and through bridge wall lumen 182 and provide the loop 18 positioned proximally of bridge wall 158 and through which extends the pull wire 30.

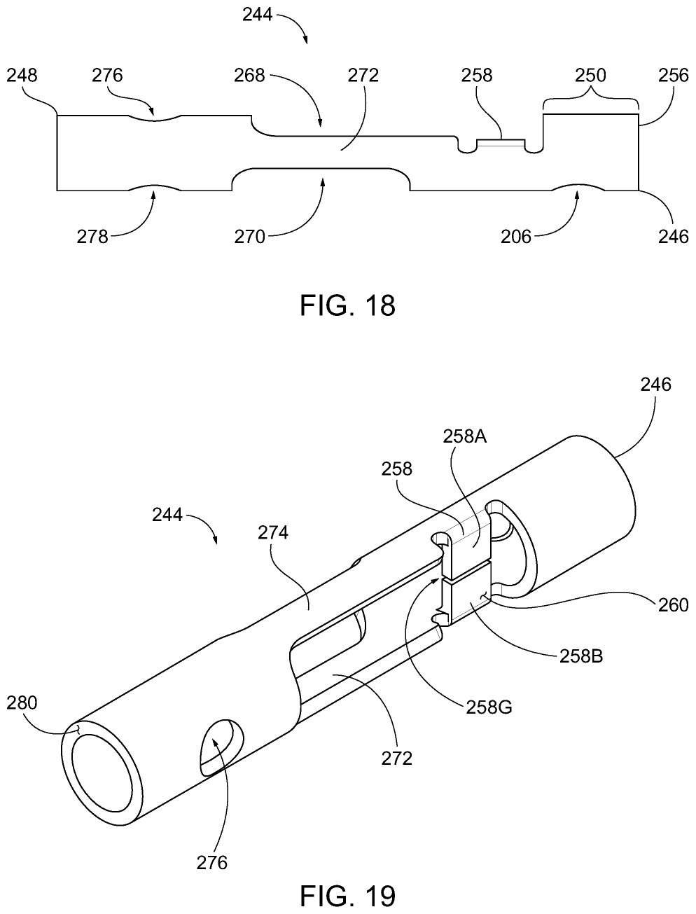

Still another bridge member structure is depicted in FIGS. 18 to 21, which further can be used in an alternative embolic coil delivery system 210 having an alternative delivery shaft/embolic coil device retention interface, as discussed further below. Bridge member 244 can have features that correspond to the features of bridge member 144, and such features of bridge member 244 are correspondingly numbered to those of bridge member 144 except in the 200 series (e.g. 244 vs. 144, 246 vs. 146, etc.) and thus the corresponding descriptions will not be repeated here. Bridge member 244 differs from bridge member 144 in that it has a longer tubular distal segment 250 that further has a ball-receiving opening 206 for receiving a retention ball as discussed below. Opening 206 can be at a position around the circumference of bridge member 244 that is aligned with the tube wall surface 284 that in part defines the bridge wall lumen 282. In this manner, when pull wire 230 extends over the upper surface 260 of bridge wall 258 and distally through tubular distal segment 250, the bridge wall 258 will urge pull wire 230 to a position within tubular distal segment 250 that is opposite the ball-receiving opening 206.

Generally, embolic coil delivery system 210 can have features that correspond to those of the version of system 10 discussed above incorporating bridge member 144, and such features of system 210 are correspondingly numbered to those of that system 10 except in the 200 series (e.g. 210 vs. 10, etc.), and thus the corresponding descriptions will not be repeated here. The coil device 214 of differs from coil device 14 of system 10 in respect of features of its retention member that cooperate with the pull wire to hold and detach the coil to and from the delivery shaft. Embolic coil device 214 has an attached ball element 200 at its proximal end that cooperates with the opening 206 of bridge member 244 and the pull wire 230 to detachably connect the coil device 214 to the delivery shaft 222. In particular, when the pull wire 230 has a portion that co-extends longitudinally with the ball element 200 as partially lodged in opening 206, and the ball element 200 is retained in the partially lodged condition because the dimensions of the ball element 200, pull wire 230, and shaft lumen 228 in the region will not allow travel of the ball element 200 so as to escape the window or opening 206 (the pull wire serving as a so-called “interference wire” in this arrangement). The detachment of the coil device 214 from the shaft 222 can be achieved by retracting the pull wire 230 proximally until its distal end 232 is positioned proximal of the ball element 200, thus releasing the ball element for travel distally through lumen 228 and out of the end 224 of the shaft 222.

In the illustrated embodiment, the ball element 200 is attached to a stem 202 that in turn is attached to an eyelet 204. The proximally located loop 218 formed by the filament material 220 is positioned through the eyelet 204. It will be understood that the filament material 220 is connected to the windings 216 of the coil device 214 as discussed above and elsewhere herein in connection with windings 16 of coil device 14 and filament material 20. It will also be understood that system 210 can be used to deliver coil device 214 to a target location and to detach coil device 214 at such location in a fashion that corresponds to the descriptions herein for system 10 and coil 14.

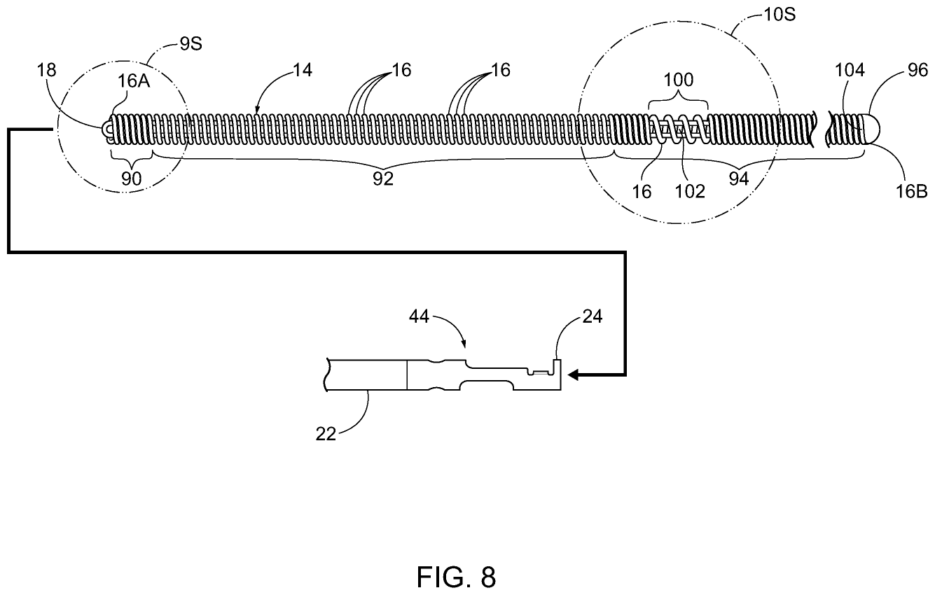

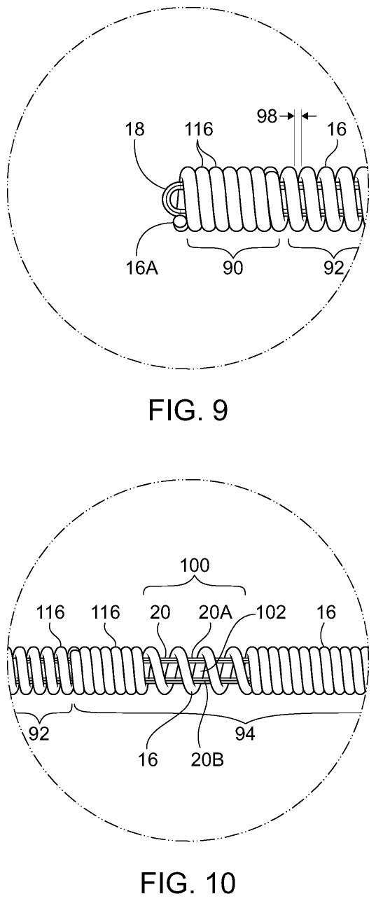

Embolic coil systems disclosed herein can have features of the embolic coil device, and of its condition as detachably connected to the delivery shaft, that facilitate a reliable detachment of the coil device from the shaft. Referring now again to FIGS. 1 to 12 and the system 10 described in conjunction therewith, the embolic coil device 14 in a relaxed (unstressed) condition (see e.g. FIGS. 8-10) has at least a segment 92 in which the windings 116 of the device are open windings wherein the surfaces of adjacent windings are longitudinally spaced from one another. Such longitudinal spacing can be a distance 98 (FIG. 9), which can in some forms be equal to at least 10% of the diameter of the wire of the windings 116, or at least 20% of such diameter, and typically in the range of about 10% to about 200%, or about 20% to about 100%, of such diameter. In addition or alternatively, adjacent windings of the coil windings in segment 92, in the relaxed (longitudinally extended) condition, can be spaced a distance from one another in the range of about 0.0005 to about 0.15 mm. The open windings of coil device 14 are configured to act as a resilient, longitudinally compressible spring segment that can be longitudinally compressed to more closely approximate their adjacent surfaces and thereby shorten the coil device 14, and that when released from such compression resiliently expand to move their adjacent surfaces away from one another and lengthen the coil device 14, for example with the open windings resiliently expanding at least toward, and potentially to, their original open and spaced condition.

To connect the coil device 14 to the shaft 22, the loop 18 can be passed through the end 46 of the bridge member 44 that provides the end 24 of the shaft 22, and forced proximally through the bridge wall lumen 82 to a position proximal of bridge wall 58. This can cause the proximal end 16A of the coil windings 16 to first contact the distally-facing surface 56 of the bridge member 44 whereupon continued proximal movement of the loop 18 resiliently compresses the open windings in segment 92 of coil device. The distal end 32 of the pull wire 30 can then be passed in a distally-directed movement through the loop 18 to connect the coil device 14 to the delivery shaft 22, with the open windings of segment 92 retained in a resiliently compressed condition. In this detachably connected state, the proximal end 16A of the coil windings 116 exerts a proximally-directed longitudinal force on the distal surface 56 of the bridge member 44. When, as discussed above, the pull wire 30 is thereafter retracted proximally to release loop 18 for detachment of the coil device 14, the resilient expansion of the opening windings of segment 92 can urge distal travel of the loop 18 through bridge wall lumen 82 and out of the end 24 of the delivery shaft 22. This can facilitate a more reliable detachment of the device 14 from the shaft 22. In the detachably connected condition of the embolic coil device 14 to the shaft 22, the distal end 32 of the pull wire 30 is positioned distal of the loop 18, preferably distal of the bridge wall 58, and even more preferably distal of the distal end 24 of the delivery shaft 22 (e.g. positioned within the lumen defined by coil windings 116). In addition or alternatively, in some forms, the distal end 32 of the pull wire 30 may be retracted at least about 1 mm, or at least about 3 mm, and typically in the range of about 1 mm to about 10 mm, to cause detachment of the embolic coil device 14. It will be understood that corresponding embolic coil connection operations can be conducted with delivery shafts having the other bridge members disclosed herein.

Any suitable length segment and position of open windings of coil device 14 that function resiliently as discussed above can be utilized. In the illustrated embodiment, a preferred arrangement is depicted in which a first, proximal-most segment 90 of coil device 14 has windings 116 that are relatively closed compared to the windings 116 of segment 92 (having adjacent windings contacting one another and/or having a smaller longitudinal spacing than the windings of segment 92). The proximally-positioned relatively closed winding segment 90 facilitates a reliable interface between the proximal end 16A of coil windings 116 and the distally-facing surface 56 of the bridge member 44. The illustrated embolic coil device 14 also has a distal segment 94 having windings 116 that are relatively closed compared to the windings 116 of segment 92 (again, having adjacent windings contacting one another and/or having a smaller longitudinal spacing than the windings of segment 92). In other embodiments, all of the windings of the embolic coil device can be open, and thus the entire length of the windings can participate in the resilient compression and expansion functions noted above, or the open winding segment can be positioned more proximally or more distally on the coil device 14 than that presently depicted, or multiple such open winding segments can be provided by the coil device 14. It will be preferred that the coil device 14 include such a resilient open winding segment (e.g. segment 92) in a region at or near the proximal end 16A of the coil windings 16, for example with the proximal end of the resilient open winding segment being within about 10 mm of proximal end 16A, for example where the proximal end of the resilient open winding segment is at proximal end 16A or is spaced distally from proximal end 16A a distance of about 0.2 to about 10 mm or in some forms about 0.5 to about 5 mm. The embolic coil device 14 also includes an element 96, such as a spherical or hemispherical member, attached to the distal end 16B of the coil windings 16 and providing a smooth distal end to the device 14.

The longitudinal length of the resilient open winding segment 92 or any other resilient open winding segment as described herein can be any suitable longitudinal length. In some forms, such longitudinal length will be at least about 1 mm, or at least about 2 mm, and typically in the range of about 1 mm to about 15 mm, more typically in the range of about 2 mm to about 8 mm.

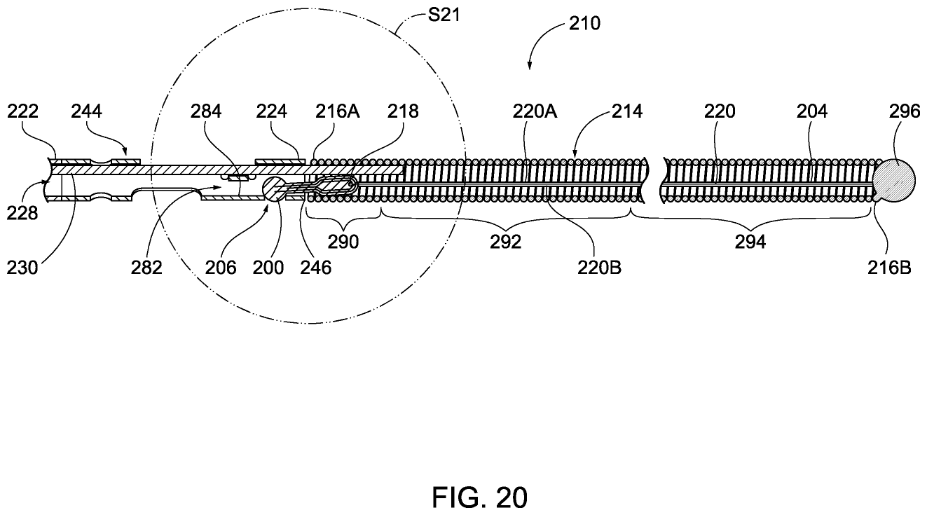

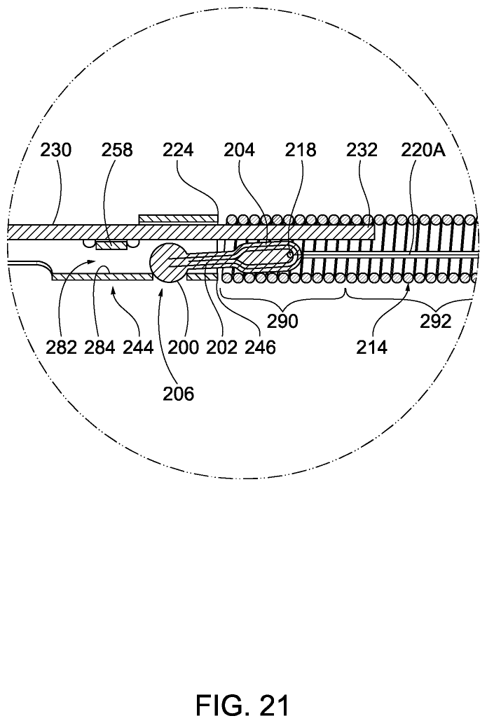

While the above discussions have focused upon the system depicted in FIGS. 1 to 12, it will be understood that the system with the alternative bridge member described in connection with FIGS. 13 to 17, and the system 210 of FIGS. 18 to 21, can also be equipped with the above-described resilient open winding features on their embolic coil devices. Still other systems within the scope of the present descriptions can also include these features. In connection with system 210 and with particular reference to FIGS. 20 and 21, it is seen that the embolic coil device 214 includes a relatively closed winding segment 290, an open winding segment 292 and a relatively closed winding segment 94, corresponding to segments 90, 92 and 94 described in conjunction with FIGS. 1 to 12. In the detachment of the coil device 214 from the shaft 222 of system 210, upon proximal movement of the distal end 232 of pull wire 230 past ball element 200 to release it from opening 206, the resilient expansion of the opening windings of segment 292 can urge distal travel of the ball 200 out of the end 224 of the delivery shaft 222. Again, this can facilitate a more reliable detachment of the coil device 214 from the shaft 222.

Referring to FIGS. 8 and 10, in preferred forms of the coil device 14 and systems including it, the filament 20 is connected to the coil windings 116 of the coil at location 100 that is just distal to the distal end of open winding segment 92, for example within about 3 mm, or within about 1 mm, of the distal end of open winding segment 92. For purposes of illustration, FIGS. 8 and 10 show location 100 having its coil windings resiliently forced open, as can be done to apply an adhesive 102 or effect another attachment of filament 20 to the coil windings. It will be understood that the particular form of attachment of the filament to the coil windings 116 may vary depending on the materials used in the filament 20 and/or coil windings 116, for example the attachment may be achieved by a solder or weld or a bonding agent such as adhesive or glue in various embodiments. After achieving the attachment, the windings at location 100 can be released to return to a more closed condition. In other forms, the coil windings at location 100 can be in a relatively open condition as depicted in FIGS. 8 and 10 in a relaxed state. Attachment of the filament 20 to the coil windings at location 100 provides a feature wherein the coil windings 116 distal of location 100 are not subjected to the compression forces discussed above in connection with the loading and detachably connected condition of the coil device 14. This is because proximal urging of the loop 18 pulls on the coil windings 116 at position 100 but not on the coil windings 116 distal of location 100. In some embodiments the filament 20 may terminate at location 100. In preferred forms, the filament continues to the distal end of embolic coil device 14 and is also attached to the coil windings at location 104. The filament can thus provide stretch resistance to the embolic coil device 14 between locations 100 and 104, for example during delivery operations prior to detachment and/or after detachment at the target site. It will be understood as well that the segment of filament 20 extending between location 100 and loop 18 will, in the detachably connected condition of the embolic coil device (e.g. during maneuvering to or at the target site for coil delivery), provide stretch resistance to the length of coil device 14 positioned proximally of location 100.

It will be understood that the above-discussed attachment features of the filament 20 to the coil windings 116 of embolic coil device 14, including the protection of coil windings distal of attachment location 100 against compression forces and/or the provision of stretch resistance by the filament 20, can also be incorporated in the coil delivery system with the alternative bridge member described in connection with FIGS. 13 to 17, in the system 210 of FIGS. 18 to 21, and in other systems within the scope of the descriptions herein.

The delivery systems 10,210 described herein can be used to deliver the coil device 14,214 to an aneurysm in a blood vessel. In doing so, the catheter 300 (e.g. microcatheter) can be positioned within the vessel so as to place its distal end 302 within the aneurysm. The delivery system 10,210 including the delivery shaft 22,222 and the detachably connected coil device 14,214 is then advanced through the catheter 300. The system 10,210 may be pre-loaded in an introducer sheath or the like (not shown). The system 10,210 is then advanced to place the coil device 14,214 in the aneurysm. One or more radiopaque markers located on the delivery system 10,210 and/or catheter 300 may be used to aid the physician in positioning the system 10,210 for deployment of the coil device 14,214. The coil device 14,214 can then be released into the aneurysm by proximal retraction of the pull wire 30,230. Where the system 10,210 includes an arrangement for protecting against unintended release of the coil device 14,214 as discussed above, prior to proximal retraction of the pull wire 30,230, the shaft 22,222 is broken at location 40 by applying bending forces to the shaft 22,222 across location 40.

### ENUMERATED LISTING OF CERTAIN EMBODIMENTS HEREIN

The following provides a non-limiting, enumerated listing of certain Embodiments herein.

1. A system for delivering an embolic coil device, comprising:
a flexible elongate delivery shaft having a distal region;
an embolic coil device detachably connected to the distal region of the elongate delivery shaft with coil windings of the embolic coil device in a resiliently longitudinally compressed condition; and
wherein upon detachment of the embolic coil device from the elongate delivery shaft the coil windings resiliently move to a longitudinally extended condition, wherein the coil windings in the longitudinally extended condition are more open than they are in the resiliently longitudinally compressed condition.

2. The system of Embodiment 1, wherein the embolic coil device is detachably connected to the distal region by a retention member of the embolic coil device interfacing with a pull wire extending through a lumen of the elongate delivery shaft, and wherein proximal retraction of the pull wire causes detachment of the embolic coil device from the elongate delivery shaft.

3. The system of Embodiment 2, wherein the retention member includes a ball and the delivery shaft defines a ball-receiving opening, wherein the ball held in the window by the pull wire until said proximal retraction.

4. The system of Embodiment 3, wherein the ball is attached to a filament extending into a lumen of the embolic coil device, preferably wherein the filament provides stretch resistance to the embolic coil device at least until said detachment.

5. The system of Embodiment 2, wherein the retention member includes a loop and the pull wire extends through the loop.

6. The system of Embodiment 5, wherein the loop is defined by a filament extending into a lumen of the embolic coil device, preferably wherein the filament provides stretch resistance to the embolic coil device at least until said detachment.

7. The system of any preceding Embodiment, wherein said windings are of a first winding segment, the first winding segment including only a portion of the coil windings of the embolic coil device.

8. The system of Embodiment 7, wherein said first winding segment is positioned in a proximal region of the embolic coil device.

9. The system of Embodiment 7 or 8, wherein the first winding segment is flanked by a flanking winding segment positioned proximal to the first winding segment, wherein the flanking winding segment has relatively closed coil windings as compared to the first winding segment.

10. The system of Embodiment 7 or 8, wherein the first winding segment is flanked by a flanking winding segment positioned distal to the first winding segment, wherein the flanking winding segment has relatively closed coil windings as compared to the first winding segment.

11. The system of Embodiment 7 or 8, wherein the first winding segment is flanked by a second winding segment positioned proximal to the first winding segment and a third winding segment positioned distal to the first winding segment, wherein the second winding segment and the third winding segment both have relatively closed coil windings as compared to the first winding segment.

12. The system of Embodiment 10, wherein the flanking winding segment extends from a distal end of the first winding segment to a distal end of the coil windings of the embolic coil device.

13. The system of Embodiment 11, wherein the third winding segment extends from a distal end of the first winding segment to a distal end of the coil windings of the embolic coil device.

14. The system of any one of Embodiments 7 to 13, wherein the first winding segment has a length of at least about 1 mm or in the range of about 1 mm to about 15 mm.

15. The system of any one of Embodiments 1 to 6, wherein the coil windings include all of the coil windings of the embolic coil device.

16. The system of any one of Embodiments 1 to 6 or 15, wherein adjacent windings of the coil windings in the longitudinally extended condition are spaced a distance from one another that equal to is at least 10% of the diameter of a wire forming the coil windings, more preferably at least 20% of said diameter.

17. The system of any one of Embodiments 1 to 6 and 15 to 16, wherein adjacent windings of the coil windings in the longitudinally extended condition are longitudinally spaced a distance from one another in the range of about 0.0005 mm to about 0.15 mm.

18. The system of any one of Embodiments 7 to 14, wherein adjacent windings of the coil windings in the first winding segment, in the longitudinally extended condition, are spaced a distance from one another that equal to is at least 10% of the diameter of a wire forming the coil windings, more preferably at least 20% of said diameter.

19. The system of any one of Embodiments 7 to 14 and 18, wherein adjacent windings of the coil windings in the first winding segment, in the longitudinally extended condition, are longitudinally spaced a distance from one another in the range of about 0.0005 to about 0.15 mm.

20. An embolic coil device for detachable connection to an elongate delivery shaft of an embolic coil delivery system, comprising:
coil windings having a longitudinally extended condition in a relaxed condition of the embolic coil device, the coil windings being resiliently compressible to a longitudinally compressed condition for attachment to the shaft and configured to resiliently move to or toward the longitudinally extended condition upon detachment from the shaft, wherein the coil windings in the longitudinally extended condition are more open than they are in the longitudinally compressed condition; and
a retention member attached to the coil windings and configured to cooperate with the shaft in a detachable connection of the embolic coil device to the shaft.

21. The device of Embodiment 20, wherein the retention member includes a ball.

22. The device of Embodiment 21, wherein the ball is attached to a filament extending into a lumen of the embolic coil device, preferably wherein the filament is configured to provide stretch resistance to the embolic coil device at least until said detachment.

23. The device of Embodiment 20, wherein the retention member includes a loop.

24. The device of Embodiment 23, wherein the loop is defined by a filament extending into a lumen of the embolic coil device, preferably wherein the filament is configured to provide stretch resistance to the embolic coil device at least until said detachment.

25. The device of any one of Embodiments 20 to 24, wherein said windings are of a first winding segment, the first winding segment including only a portion of the coil windings of the embolic coil device.

26. The device of Embodiment 25, wherein said first winding segment is positioned in a proximal region of the embolic coil device.

27. The device of Embodiment 25 or 26, wherein the first winding segment is flanked by a flanking winding segment positioned proximal to the first winding segment, wherein the flanking winding segment positioned proximal to the first winding segment has relatively closed coil windings as compared to the first winding segment.

28. The device of Embodiment 25 or 26, wherein the first winding segment is flanked by a flanking winding segment positioned distal to the first winding segment, wherein the flanking winding segment positioned distal to the first winding segment has relatively closed coil windings as compared to the first winding segment.

29. The device of Embodiment 25 or 26, wherein the first winding segment is flanked by a second winding segment positioned proximal to the first winding segment and a third winding segment positioned distal to the first winding segment, wherein the second winding segment and the third winding segment both have relatively closed coil windings as compared to the first winding segment.

30. The device of Embodiment 28, wherein the flanking winding segment extends from a distal end of the first winding segment to a distal end of the coil windings of the embolic coil device.

31. The device of Embodiment 29, wherein the third winding segment extends from a distal end of the first winding segment to a distal end of the coil windings of the embolic coil device.

32. The device of any one of Embodiments 25 to 31, wherein the first winding segment has a length of at least about 1 mm or in the range of about 1 mm to about 15 mm.

33. The device of any one of Embodiments 20 to 24, wherein the coil windings include all of the coil windings of the embolic coil device.

34. The device of any one of Embodiments 20 to 24 or 33, wherein adjacent windings of the coil windings in the longitudinally extended condition are longitudinally spaced a distance from one another that equal to is at least 10% of the diameter of a wire forming the coil windings, more preferably at least 20% of said diameter.

35. The device of any one of Embodiments 20 to 24 and 33 to 34, wherein adjacent windings of the coil windings in the longitudinally extended condition are longitudinally spaced a distance from one another in the range of about 0.0005 to about 0.15 mm.

36. The device of any one of Embodiments 25 to 32, wherein adjacent windings of the coil windings in the first winding segment, in the longitudinally extended condition, are longitudinally spaced a distance from one another that equal to is at least 10% of the diameter of a wire forming the coil windings, more preferably at least 20% of said diameter.

37. The device of any one of Embodiments 25 to 32 and 36, wherein adjacent windings of the coil windings in the first winding segment, in the longitudinally extended condition, are longitudinally spaced a distance from one another in the range of about 0.0005 to about 0.15 mm.

38. The system of any one of Embodiments 2 to 19, wherein the elongate delivery shaft has a coil detachment interface at the distal region of the flexible elongate delivery shaft, the coil detachment interface including a distal tubular segment and a bridge segment proximal of the distal tubular segment, optionally wherein the bridge segment includes a bridge wall defining an upper surface and a lower surface, the bridge segment defining a lumen between the lower surface of the bridge wall and a bottom wall of the bridge segment.

39. The system of Embodiment 38, wherein the bridge wall defines a proximally-facing edge spanning between the upper surface and the lower surface and a distally-facing edge spanning between the upper surface and the lower surface.

40. The system of Embodiment 38 or 39, wherein the bridge wall is generally planar or defines at least one curved wall portion and/or wherein the bridge wall is rectangularly-shaped in longitudinal cross-section.

41. The system of any one of Embodiments 38 to 40, wherein the bridge wall defines a first curved portion and a second curved portion.

42. The system of Embodiment 41, wherein the first curved portion and the second curved portion each define a concave surface of the lower surface of the bridge wall and an opposite convex surface of the upper surface of the bridge wall.

43. The system of Embodiment 42, wherein the bridge wall defines a third curved portion intermediate and connecting the first and second curved portions.

44. The system of Embodiment 43, wherein the third curved portion defines a convex surface of the lower surface of the bridge wall and an opposite concave surface of the upper surface of the bridge wall.

45. The system of any one of Embodiments 38 to 44, wherein the bridge wall extends generally perpendicular to a longitudinal axis of the elongate delivery shaft.

46. The system of any one of Embodiments 38 to 45, wherein the elongate delivery shaft includes a bridge member attached to a distal end of a length of tube, preferably metal hypotube, wherein the bridge member provides the distal tubular segment and the bridge segment of the coil detachment interface; optionally wherein the bridge member includes a bridge component extending transverse to a longitudinal axis of the bridge member and defines at least one sidewall opening having an opening portion occurring proximal and proximate to a proximal surface of the bridge component; and/or optionally wherein the delivery shaft has a shaft segment in a proximal region of the delivery shaft that is configured for selective break and separation of the delivery shaft at the shaft segment in response to bending forces applied across the shaft segment, and the pull wire is attached to the delivery shaft at a location proximal to the shaft segment.

47. The system of Embodiment 46, wherein said bridge segment is an intermediate segment of said bridge member, the bridge member also including tubular segment proximal of the bridge segment and defining a proximally-facing circumferential surface, with the bridge member attached to the distal end of the length of tube, preferably metal hypotube, at the proximally-facing circumferential surface.

48. The system of Embodiment 46 or 47, wherein the bridge member is a monolithic structure formed from a single length of tube.

49. The system of any one of Embodiments 38 to 48, wherein the retention member comprises a ball at a proximal end of the embolic coil device.

50. The system of Embodiment 49, wherein the elongate delivery shaft defines a ball-receiving opening, and wherein the ball is held in the ball-receiving opening by the pull wire until said proximal retraction of the pull wire.

51. The system of any one of Embodiments 46 to 48, wherein the retention member comprises a ball at a proximal end of the embolic coil device, wherein the distal tubular segment of the bridge member defines a ball-receiving opening, and wherein the ball is held in the ball receiving opening by the pull wire until said proximal retraction of the pull wire.

52. The system of Embodiment 51, wherein the ball-receiving opening is defined in a sidewall of the distal tubular segment of the bridge member.

53. The system of any one of Embodiments 38 to 52, wherein the pull wire extends in a path over the upper surface of the bridge wall.

54. The system of any one of Embodiments 38 to 48, wherein the retention member comprises a loop attached to the embolic coil.

55. The system of Embodiment 54, wherein the pull wire extends through the loop until said proximal translation of the pull wire.

56. The system of Embodiment 55, wherein a proximal end of the loop is held at a position proximal of a bridge component, preferably a/the bridge wall, until said proximal translation of the pull wire.

57. The system of Embodiment 56, wherein the retention member extends through the lumen defined by the bridge segment and the pull wire extends in a path through the loop and then over the upper surface of the bridge wall.

58. The system of any one of Embodiments 38 to 57, wherein the distal tubular segment has a distal-most end that defines a distally-facing full circumferential surface, and wherein a proximal end of coil windings of the embolic coil device abuts the distally-facing full circumferential surface.

59. The system of any one of Embodiments 38 to 58, wherein the pull wire has a distal end positioned distal of a/the bridge component, preferably a/the bridge wall.

60. The system of Embodiment 59, wherein the distal end of the pull wire is positioned distal of the distal tubular segment.

61. A system for delivering an embolic coil device, comprising:
a flexible elongate delivery shaft defining a lumen and having a distal region;
a coil detachment interface at the distal region of the flexible elongate delivery shaft, the coil detachment interface including a distal tubular segment and a bridge segment proximal of the distal tubular segment, the bridge segment including a bridge wall or other bridge component defining an upper surface and a lower surface, the bridge segment defining a lumen between the lower surface of the bridge wall or other bridge component and a bottom wall of the bridge segment; optionally wherein the bridge segment defines a sidewall opening having an opening portion occurring proximal and proximate to a proximal surface of the bridge wall or other bridge component;
an embolic coil device detachably connected to the distal region of the elongate delivery shaft;
a pull wire extending through the lumen of the elongate delivery shaft and interfacing with a retention member of the embolic coil device; and
wherein proximal retraction of the pull wire causes detachment of the embolic coil device from the elongate delivery shaft.

62. The system of Embodiment 61, wherein the bridge wall defines a proximally-facing edge spanning between the upper surface and the lower surface and a distally-facing edge spanning between the upper surface and the lower surface.

63. The system of Embodiment 61 or 62, wherein the bridge wall is generally planar or defines at least one curved wall portion.

64. The system of Embodiment 61 or 62, wherein the bridge wall defines a first curved portion and a second curved portion.

65. The system of Embodiment 64, wherein the first curved portion and the second curved portion each define a concave surface of the lower surface of the bridge wall and an opposite convex surface of the upper surface of the bridge wall.

66. The system of Embodiment 65, wherein the bridge wall defines a third curved portion intermediate and connecting the first and second curved portions.

67. The system of Embodiment 66, wherein the third curved portion defines a convex surface of the lower surface of the bridge wall and an opposite concave surface of the upper surface of the bridge wall.

68. The system of any one of Embodiments 61 to 67, wherein the bridge wall extends generally perpendicular to a longitudinal axis of the elongate delivery shaft.

69. The system of any one of Embodiments 61 to 68, wherein the elongate delivery shaft includes a bridge member attached to a distal end of a length of tube, optionally metal hypotube, wherein the bridge member provides the distal tubular segment and the bridge segment of the coil detachment interface; and optionally wherein the delivery shaft has a shaft segment in a proximal region of the delivery shaft that is configured for selective break and separation of the delivery shaft at the shaft segment in response to bending forces applied across the shaft segment, and the pull wire is attached to the delivery shaft at a location proximal to the shaft segment.

70. The system of Embodiment 69, wherein said bridge segment is an intermediate segment of said bridge member, the bridge member also including tubular segment proximal of the bridge segment and defining a proximally-facing circumferential surface, with the bridge member attached to the distal end of the length of tube, preferably metal hypotube, at the proximally-facing circumferential surface.

71. The system of Embodiment 69 or 70, wherein the bridge member is a monolithic structure formed from a single length of tube.

72. The system of any one of Embodiments 61 to 71 wherein the retention member comprises a ball at a proximal end of the embolic coil device.

73. The system of Embodiment 72, wherein the elongate delivery shaft defines a ball-receiving opening, and wherein the ball is held in the ball receiving opening by the pull wire until said proximal retraction of the pull wire.

74. The system of an one of Embodiments 69 to 71, wherein the retention member comprises a ball at a proximal end of the embolic coil device, wherein the distal tubular segment of the bridge member defines a ball-receiving opening, and wherein the ball is held in the ball receiving opening by the pull wire until said proximal retraction of the pull wire.

75. The system of Embodiment 74, wherein the ball-receiving opening is defined in a sidewall of the distal tubular segment of the bridge member.

76. The system of any one of Embodiments 61 to 75, wherein the pull wire extends in a path over the upper surface of the bridge wall.

77. The system of any one of Embodiments 61 to 71, wherein the retention member comprises a loop attached to the embolic coil.

78. The system of Embodiment 77, wherein the pull wire extends through the loop until said proximal translation of the pull wire.

79. The system of Embodiment 78, wherein a proximal end of the loop is held at a position proximal of the bridge wall until said proximal translation of the pull wire.

80. The system of Embodiment 79, wherein the retention member extends through the lumen defined by the bridge segment and the pull wire extends in a path through the loop and then over the upper surface of the bridge wall.

81. The system of any one of Embodiments 61 to 80, wherein the distal tubular segment has a distal-most end that defines a distally-facing full circumferential surface, and wherein a proximal end of coil windings of the embolic coil device abuts the distally-facing full circumferential surface.

82. The system of any one of Embodiments 61 to 81, wherein the pull wire has a distal end positioned distal of the bridge wall.

83. The system of Embodiment 82, wherein the distal end of the pull wire is positioned distal of the distal tubular segment.

84. A bridge member useful in a detachment interface of an embolic coil device delivery system, comprising:
a distal tubular segment, a proximal tubular segment, and an intermediate segment between the distal tubular segment and the proximal tubular segment, wherein (i) the intermediate segment includes a bridge wall defining an upper surface and a lower surface, the intermediate segment defining a lumen between the lower surface of the bridge wall and a bottom wall of the bridge segment; or (ii) the intermediate segment includes a bridge component extending transverse to a longitudinal axis of the bridge member and the intermediate segment defines at least one sidewall opening having an opening portion occurring proximal and proximate to a proximal surface of the bridge component, preferably wherein (a) the at least one sidewall opening has a maximum width in a direction perpendicular to the longitudinal axis of bridge member that is least 40% of a corresponding width of the bridge member that longitudinally coextends with the at least one sidewall opening, (b) the at least one sidewall opening has a maximum length in the direction of the longitudinal axis of the bridge member that is at least equal to the maximum width of the at least one sidewall opening, and/or (c) said opening portion is longitudinally proximal of the proximal surface of the bridge component by a distance no greater than 200% of the corresponding width of the bridge member that longitudinally coextends with the at least one opening.

85. The bridge member of Embodiment 84, wherein the bridge wall defines a proximally-facing edge spanning between the upper surface and the lower surface and a distally-facing edge spanning between the upper surface and the lower surface.

86. The bridge member of Embodiment 84, wherein the bridge wall is generally planar or defines at least one curved wall portion.

87. The bridge member of Embodiment 84 or 85, wherein the bridge wall defines a first curved portion and a second curved portion.

88. The bridge member of Embodiment 87, wherein the first curved portion and the second curved portion each define a concave surface of the lower surface of the bridge wall and an opposite convex surface of the upper surface of the bridge wall.

89. The bridge member of Embodiment 88, wherein the bridge wall defines a third curved portion intermediate and connecting the first and second curved portions.

90. The bridge member of Embodiment 89, wherein the third curved portion defines a convex surface of the lower surface of the bridge wall and an opposite concave surface of the upper surface of the bridge wall.

91. The bridge member of any one of Embodiments 84 to 90, wherein the bridge wall extends generally perpendicular to a longitudinal axis of the bridge member.

92. A flexible elongate delivery shaft useful in a system for delivering an embolic coil device, comprising a bridge member according to any one of Embodiments 84 to 91 attached to a distal end of a length of tube, preferably a metal hypotube.

93. The flexible elongate delivery shaft of Embodiment 92, having a shaft segment in a proximal region of the delivery shaft that is configured for selective break and separation of the delivery shaft at the shaft segment in response to bending forces applied across the shaft segment.

94. The flexible elongate delivery shaft of Embodiment 92, in combination with a pull wire extending through a lumen of the elongate delivery shaft; and also optionally in combination with a coil device detachably connected to the flexible elongate delivery shaft.

95. The combination of Embodiment 94, wherein the delivery shaft has a shaft segment in a proximal region of the delivery shaft that is configured for selective break and separation of the delivery shaft at the shaft segment in response to bending forces applied across the shaft segment, and wherein the pull wire is attached to the delivery shaft at a location proximal to the shaft segment.

96. The system of any one of Embodiments 61 to 83, wherein:
the embolic coil device has coil windings of the embolic coil device in a resiliently longitudinally compressed condition; and
upon detachment of the embolic coil device from the elongate delivery shaft the coil windings resiliently move to a longitudinally extended condition, wherein the coil windings in the longitudinally extended condition are more open than they are in the resiliently longitudinally compressed condition.

97. The system of Embodiment 96, wherein said windings are of a first winding segment, the first winding segment including only a portion of the coil windings of the embolic coil device.

98. The system of Embodiment 97, wherein said first winding segment is positioned in a proximal region of the embolic coil device.

99. The system of Embodiment 97 or 98, wherein the first winding segment is flanked by a flanking winding segment positioned proximal to the first winding segment, wherein the flanking winding segment positioned proximal to the first winding segment has relatively closed coil windings as compared to the first winding segment.

100. The system of Embodiment 97 or 98, wherein the first winding segment is flanked by a flanking winding segment positioned distal to the first winding segment, wherein the flanking winding segment positioned distal to the first winding segment has relatively closed coil windings as compared to the first winding segment.

101. The system of Embodiment 97 or 98, wherein the first winding segment is flanked by a second winding segment positioned proximal to the first winding segment and a third winding segment positioned distal to the first winding segment, wherein the second winding segment and the third winding segment both have relatively closed coil windings as compared to the first winding segment.

102. The system of Embodiment 100, wherein the flanking winding segment positioned distal to the first winding segment extends from a distal end of the first winding segment to a distal end of the coil windings of the embolic coil device.

103. The system of Embodiment 101, wherein the third winding segment extends from a distal end of the first winding segment to a distal end of the coil windings of the embolic coil device.

104. The system of any one of Embodiments 97 to 103, wherein the first winding segment has a length of at least 1 mm or in the range of about 1 mm to about 15 mm.

105. The system of Embodiment 96, wherein the coil windings include all of the coil windings of the embolic coil device.

106. The system of any one of Embodiments 96 to 105, wherein adjacent windings of the coil windings in the longitudinally extended condition are spaced a distance from one another that equal to is at least 10% of the diameter of a wire forming the adjacent coil windings, more preferably at least 20% of said diameter.

107. The system of any one of Embodiments 96 to 106, wherein adjacent windings of the coil windings in the longitudinally extended condition are spaced a distance from one another in the range of about 0.005 to about 0.15 mm.

The uses of the terms “a” and “an” and “the” and similar references herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the products or methods defined by the claims.

While embodiments of the disclosure have been illustrated and described in detail in the drawings and foregoing description, the same is to be considered as illustrative and not restrictive in character, it being understood that only some embodiments have been shown and described and that all changes and modifications that come within the spirit of the disclosures herein are desired to be protected.

1. A system for delivering an embolic coil device, comprising:
a flexible elongate delivery shaft having a distal region;
an embolic coil device detachably connected to the distal region of the elongate delivery shaft with coil windings of the embolic coil device in a resiliently longitudinally compressed condition; and
wherein upon detachment of the embolic coil device from the elongate delivery shaft the coil windings resiliently move to a longitudinally extended condition, wherein the coil windings in the longitudinally extended condition are more open than they are in the resiliently longitudinally compressed condition.

2. The system of claim 1, wherein the embolic coil device is detachably connected to the distal region by a retention member of the embolic coil device interfacing with a pull wire extending through a lumen of the elongate delivery shaft, and wherein proximal retraction of the pull wire causes detachment of the embolic coil device from the elongate delivery shaft.

3. The system of claim 2, wherein the retention member includes a ball and the delivery shaft defines a ball-receiving opening, wherein the ball held in the window by the pull wire until said proximal retraction.

4. The system of claim 3, wherein the ball is attached to a filament extending into a lumen of the embolic coil device.

5. The system of claim 2, wherein the retention member includes a loop and the pull wire extends through the loop.

6. The system of claim 5, wherein the loop is defined by a filament extending into a lumen of the embolic coil device.

7. The system of claim 1, wherein said windings are of a first winding segment, the first winding segment including only a portion of the coil windings of the embolic coil device.

8. The system of claim 7, wherein said first winding segment is positioned in a proximal region of the embolic coil device.

9. The system of claim 7, wherein the first winding segment is flanked by a flanking winding segment positioned proximal to the first winding segment, wherein the second winding segment has relatively closed coil windings as compared to the first winding segment.

10. The system of claim 7, wherein the first winding segment is flanked by a flanking winding segment positioned distal to the first winding segment, wherein the flanking winding segment has relatively closed coil windings as compared to the first winding segment.

11-13. (canceled)

14. The system of claim 7, wherein the first winding segment has a length of at least about 1 mm.

15. The system of claim 1, wherein the coil windings include all of the coil windings of the embolic coil device.

16. The system of claim 1, wherein adjacent windings of the coil windings in the longitudinally extended condition are spaced a distance from one another that equal to is at least 10% of the diameter of a wire forming the coil windings.

17. The system of claim 1, wherein adjacent windings of the coil windings in the longitudinally extended condition are longitudinally spaced a distance from one another in the range of about 0.0005 mm to about 0.15 mm.

18-19. (canceled)

20. An embolic coil device for detachable connection to an elongate delivery shaft of an embolic coil delivery system, comprising:
coil windings having a longitudinally extended condition in a relaxed condition of the embolic coil device, the coil windings being resiliently compressible to a longitudinally compressed condition for attachment to the shaft and configured to resiliently move to or toward the longitudinally extended condition upon detachment from the shaft, wherein the coil windings in the longitudinally extended condition are more open than they are in the longitudinally compressed condition; and
a retention member attached to the coil windings and configured to cooperate with the shaft in a detachable connection of the embolic coil device to the shaft.

21. The device of claim 20, wherein the retention member includes a ball.

22. The device of claim 21, wherein the ball is attached to a filament extending into a lumen of the embolic coil device.

23. The device of claim 20, wherein the retention member includes a loop.

24. The device of claim 23, wherein the loop is defined by a filament extending into a lumen of the embolic coil device.

25. The device of claim 20, wherein said windings are of a first winding segment, the first winding segment including only a portion of the coil windings of the embolic coil device.

26-60. (canceled)
